(19) United States
(12) Patent Application Publication
(10) Pub. No.: US 2004/0122390 A1
(43) Pub. Date: Jun. 24, 2004

(54) Low evaporative superabsorbent products and composites and methods for utilizing the same

(75) Inventors: Dave Allen Soerens, Neenah, WI (US); Jian Qin, Appleton, WI (US); Eugenio Varona, Marietta, GA (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/328,441

(22) Filed: Dec. 23, 2002

(51) Int. Cl. A61F 13/15; A61F 13/20

(52) U.S. Cl. 604/367

(57) ABSTRACT

Low evaporative absorbent articles are disclosed. The low evaporative absorbent articles comprise a treatment agent in the absorbent core of the absorbent article which, upon activation, coats swollen superabsorbent particles present in the absorbent core to reduce evaporation therefrom.

### BACKGROUND OF THE INVENTION

The present invention relates to superabsorbent particle composites for use in absorbent articles such as diapers and incontinence garments. More particularly, the present invention relates to low evaporative superabsorbent particle composites which include a superabsorbent particle and a surfactant located in the interior of the superabsorbent particle which, upon activation, can diffuse to the outside surface of the superabsorbent particle and provide a coating on that surface to reduce evaporation of water therefrom. The present invention also relates to low evaporative absorbent articles which include various components, such as a fibrous network of absorbent fibers, including a treatment agent which, when activated, can coat the outside surface of swollen superabsorbent particles to reduce the evaporation of water therefrom.

Disposable absorbent articles such as diapers, incontinence garments, catamenial devices, training pants, feminine napkins, interlabial devices, and other absorbent products, are well known in the art. Typically, disposable absorbent articles include a liquid pervious topsheet that faces the wearer's body, a liquid impervious backsheet that faces the wearer's clothing, an absorbent core disposed between the liquid pervious topsheet and the backsheet, and means to keep the core in fixed relation to the wearer's body. The absorbent core typically contains a network of absorbent fibers intermixed with superabsorbent particles, commonly known as “SAPs,” which can absorb and hold many times their weight of a liquid. Common SAPs include crosslinked polyacylates such as poly(acrylic) acid. The use of SAPs allows many absorbent products to be highly absorbent without being substantially bulky.

In order to absorb and contain bodily exudates such as urine, feces or menstrual fluids, an absorbent article must occlude certain parts of a wearer's body. Occlusion of the skin by the absorbent article can, however, result in a high moisture content in the skin—absorbent article microenvironment and, potentially, lead to skin overhydration with resulting increased risk of skin irritation. Further, as skin becomes overhydrated, it becomes macerated. As a result, overhydrated skin is more susceptible to damage from abrasion due to rubbing caused by normal wearer movements (i.e. chafing). Such susceptibility to skin disorders, including diaper rash, erythema, heat rash, abrasion, pressure marks, and skin barrier loss is well known and is a problem of great concern.

The stratum corneum is the skin layer that, almost exclusively, provides the water barrier properties to the skin. As such, any environmental condition that can increase the hydration state of the stratum corneum will typically lead to skin overhydration. Occlusion by an absorbent article is a prime example of an environmental condition that can lead to skin overhydration. In particular, skin occluded by an absorbent article sees at least the following differences in its environment when compared to unoccluded skin:

(1) Available water from bodily fluids, such as urine, increases the driving force across the extra cellular lipid component of the stratum corneum (the hydrophobic component which provides the main water barrier properties to the stratum corneum) allowing the keratin enriched corneocyte components of the stratum corneum (the hydrophilic component which provides mechanical strength to the stratum corneum) to become overhydrated. Such available water can come from inadequate acquisition by the absorbent article, from rewet because the absorbent article fails to have adequate liquid retention capability, from evaporation from superabsorbent particles, or from sweat due to the occlusive nature of the absorbent article.

(2) Increased relative humidity in the void volume between the absorbent article and the skin can interfere with the natural transport of water vapor into and out of the skin. As is well known, mass transport depends on a concentration differential across a barrier. If the relative humidity on the outside of the stratum corneum becomes too high and additional water is delivered to the body side of the stratum corneum (e.g., due to an increase in ambient temperature), the water will remain in/on the skin for a longer period of time.

Once skin begins to become overhydrated, the barrier properties of the extra cellular lipid component of the stratum corneum begin to degenerate. Such degeneration results in increased overhydration, leading to compromised skin and diaper rash, as well as other potential problems.

As mentioned above, one major contributor to skin overhydration and high relative humidity inside of a diaper microenvironment is the evaporation of water from swollen SAPs contained in the absorbent core. Although these SAPs are highly absorbent and can greatly decrease the size of absorbent core structures and minimize leakage, evaporative water loss from swollen SAPs can negatively affect skin health as noted above. Furthermore, high levels of evaporation may also result in high levels of odor emanating from absorbent products such as diapers. As such, a need exists for superabsorbent particle composites, which have high levels of absorptivity of salt containing solutions, but which are also capable of retaining absorbed fluids without allowing substantial evaporation over an extended period of time, which can lead to high humidity levels and skin overhydration. Also, absorbent products comprising the low evaporative superabsorbent particle composites are desirable.

### SUMMARY OF THE INVENTION

The present invention relates to low evaporative absorbent articles such diapers, incontinence garments, feminine products, etc. The absorbent article includes a superabsorbent particle and a treatment agent. When the absorbent core of the absorbent article receives a salt-containing solution, such as urine or menses, excreted from a wearer, the treatment agent is activated and migrates to the outer surface of the superabsorbent particles in the absorbent core and coats that surface to reduce evaporation therefrom.

In one embodiment, the treatment agent can be a surfactant, which is incorporated directly into the interior, and preferably the center, of the superabsorbent particle during manufacturing. Upon urination by a wearer, the superabsorbent particles, which are typically located in the absorbent core of the absorbent article, absorb the liquid and swell, thereby activating the surfactant. Upon activation, the surfactant diffuses to the outer surface of the superabsorbent particles and forms a coating thereon which reduces the rate of evaporation from the superabsorbent particle.

In another embodiment, the treatment agent can be a cationic starch or another suitable compound, which is coated onto a component in the absorbent product, such as a fibrous network of absorbent fibers contained in the absorbent core along with superabsorbent particles. When urination or other insult occurs and the fibrous network of absorbent fibers is contacted with the excreted liquid, the treatment agent is dissolved into the liquid and contacts the outer surface of the superabsorbent particles creating a coating thereon to reduce evaporation.

Additionally, the present invention relates to methods for reducing skin overhydration due to water evaporation from swollen superabsorbent particles contained within an absorbent article worn next to the skin. In one method, a surfactant is introduced into the interior of a superabsorbent particle such that when the superabsorbent particle is contacted with urine excreted by a wearer and swells during absorption, the surfactant diffuses to the outer surface of the superabsorbent particle and coats the outer surface of the particle to reduce the rate of water evaporation from the coated outer surface.

Another method for reducing skin overhydration due to water evaporation from swollen superabsorbent particles includes introducing a treatment agent, such as a cationic starch, onto a fibrous network of absorbent fibers contained in an absorbent core of an absorbent article along with superabsorbent particles. When urine is excreted by a wearer of the absorbent product, the treatment agent is solubilized in the urine and can diffuse or migrate to the outer surface of the superabsorbent particles. This migration results in a coating of the outer surface of superabsorbent particles and reduces the evaporation of absorbed liquid from the superabsorbent particles.

Other features and advantages of this invention will be in part apparent and in part pointed out hereinafter.

### BRIEF DESCRIPTION OF THE DRAWINGS

FIG. 1 is a cross sectional view of a conventional diaper.

### DETAILED DESCRIPTION OF THE PREFERRED EMBODIMENTS

In accordance with the present invention, it has been discovered that low evaporative absorbent products comprising superabsorbent particles, such as diapers, incontinence garments, and feminine products, for example, can be manufactured utilizing a treatment agent that will reduce evaporation from the superabsorbent particles after liquid is absorbed therein. The treatment agent, upon activation, will diffuse to the outer surface of a superabsorbent particle and coat that surface during or after the superabsorbent particle has imbibed liquid and become swollen. The coating on the superabsorbent particle surface reduces the amount of liquid that can evaporate from the superabsorbent particle and can thereby reduce the tendency for skin overhydration caused by high moisture levels in the microenvironment of an absorbent product. Advantageously, when the treatment agent is a surfactant, it can be introduced into the interior of the superabsorbent particle during manufacturing such that it will migrate and coat the surface when the superabsorbent particle is substantially completely swollen (i.e., at least about 75% or more swollen) to retard evaporation of water into the absorbent article microenvironment.

The low evaporative absorbent products discussed herein can include numerous components and absorbent products well known in the art. Suitable absorbent products for use with the invention as described herein include, for example, diapers, adult incontinence garments, training pants, feminine napkins, tampons, interlabial devices, and the like. Although discussed primarily herein in combination with a diaper and urine, the low evaporative absorbent articles, superabsorbent particle composites, and related methods of the present invention are applicable to absorbent articles in general, which include superabsorbent particles used with salt-containing solutions such as urine and menses.

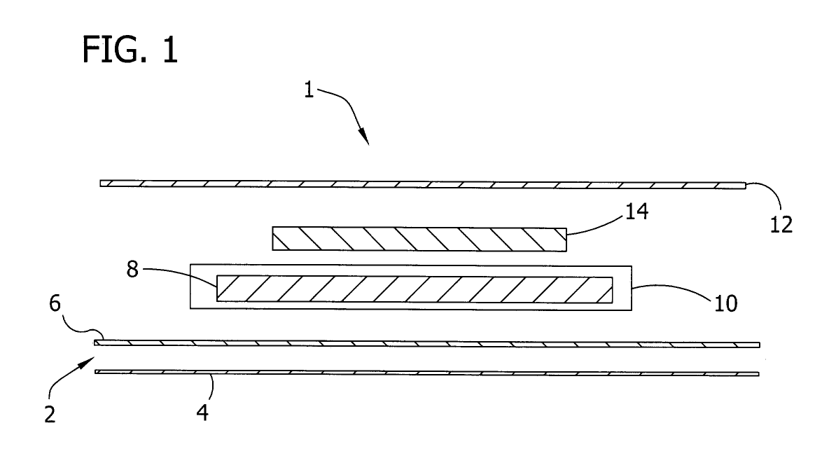

Conventional absorbent articles, such as diapers, are well known in the art and typically include numerous components, which together provide a laminated structure. As illustrated in FIG. 1, a conventional diaper 1 typically includes an outercover 2, which typically includes a liquid permeable outer layer 4 and a liquid impermeable and vapor permeable inner layer 6. The outer cover may optionally have attached thereto a loop material or “pub patch” (not shown) for receiving hook material for fastening or closing the diaper during use.

Additionally, the diaper includes an absorbent core 8 which can optionally be adhesively bonded to tissue wrap 10. Alternatively, the absorbent core need not have a tissue wrap and can simply be sandwiched between the outer cover and the bodyside liner 12. Additionally, surge management layer 14 can be sandwiched between absorbent core 8 and bodyside liner 12 to improve the fluid management properties of the diaper.

The absorbent core may have any of a number of shapes, including rectangular, I-shaped, or T-shaped and is desirably narrower in the crotch region than in the front or back regions of the diaper. The size and the absorbent capacity of the absorbent core may be selected according to the size of the intended wearer and the liquid loading imparted by the intended use of the diaper. Further, the size and the absorbent capacity of the absorbent core can be varied to accommodate various sized wearers. In addition, it has been found that the densities and/or basis weights of the absorbent core desirably has an absorbent capacity of at least about 300 grams of 0.9% (by weight) saline solution.

The absorbent core generally includes hydrophilic fibers and superabsorbent particles (also known as ionic hydrogels or ionic hydrocolloids) as described more fully below. Various types of wettable, hydrophilic fibrous materials can be used to form a fibrous network of fibers, which may form at least a portion of the absorbent core, or the entire absorbent core. Examples of suitable fibers include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material, which has a suitable hydrophilic moiety and is not readily removable from the fiber, or by sheathing the nonwettable, hydrophobic fiber with a hydrophilic polymer during or after the formation of the fiber. For the purposes of the present invention, it is contemplated that selected blends of the various types of fibers mentioned above may also be employed.

The absorbent core may include a combination of hydrophilic fibers and high-absorbency material or superabsorbent particles. Superabsorbent particles are typically cross-linked ionic polymers that are able to absorb an amount of a 0.9% (by weight) saline solution equal to at least ten times their dry weight and retain the saline solution under a moderate external pressure. However, it is understood that absorbent bodies having absorbent layers of other compositions and having dimensions other than described may be used without departing from the scope of the present invention. More specifically, the high-absorbency material in the absorbent core can be selected from natural, synthetic, and modified natural polymers and materials. The high-absorbency materials can be inorganic materials, such as silica gels, or organic compounds, such as crosslinked polymers. The term “crosslinked” refers to methods for effectively rendering normally water-soluble materials substantially water insoluble, but swellable. Such methods include, for example, physical entanglement, crystalline domains, covalent bonds, ionic complexes and associations, hydrophilic associations such as hydrogen bonding, and hydrophobic associations or Van der Waals forces.

Superabsorbent polymers can be anionic in nature (e.g., acrylate based or sulfonate based), or can be cationic in nature (e.g., a partly neutralized polyamine), and, as such, can either have positive or negative charges along the backbone of the polymer structure. Superabsorbent polymers are electrically charged in solution because various groups attached to the polymer chain easily become ionic. Examples of groups, which can become electrically charged in ionic superabsorbent polymers, include carboxylate groups and amine groups. Liquid absorbed by a superabsorbent polymer is taken directly into the molecular structure itself, and is not simply contained in pores or openings in the material from which it could be easily expressed by the application of pressure.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl amine), poly(vinyl alcohol), and mixtures and copolymers thereof. Other high-absorbency materials include mixtures of polyacids with polyamines to provide in situ neutralized superabsorbents. Further polymers suitable for use in the absorbent core include natural and modified natural polymers, such as hydrolyzed acrylonitrile-grafted starch, acrylic acid grafted starch, methyl cellulose, carboxymethyl cellulose, poly(aspartate), hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. The most commonly used superabsorbents are in the form of discrete particles or beads. However, the high absorbency material may also be in the form of fibers, flakes, rods, spheres, needles, or the like. In general, the high absorbency material is present in the absorbent core in an amount of from about 5 to about 95 percent by weight, suitably in an amount of about 30 percent by weight or more, and even more suitably in an amount of at least about 50 percent by weight or more based on a total weight of the absorbent core. An example of high-absorbency material suitable for use in the absorbent core is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

In one preferred embodiment of the present invention, the superabsorbent particles contained in the absorbent core of the absorbent garment are treated such that after absorbing a bodily fluid, such as urine or menses, and becoming substantially swollen, the superabsorbent particles release up to about 50 percent less water through evaporation back into the diaper microenvironment as compared to untreated superabsorbent particles. In order to decrease evaporation from the swollen superabsorbent particle into the diaper microenvironment, a surfactant material is introduced into the interior of the superabsorbent particle. Desirably, the surfactant material is introduced into the center of the interior of the superabsorbent particle. Methods of preparing superabsorbent particles comprising a surfactant in the interior are described in detail below.

When a superabsorbent particle includes a surfactant in its interior, the superabsorbent particle is free to absorb liquids without substantial interference from the surfactant. Once the superabsorbent particle has become substantially or completely swollen with a liquid, such as urine, the imbibed liquid contacts and activates the surfactant in the interior of the superabsorbent particle and allows the surfactant to become solvated and diffuse outward toward the outside surface of the swollen superabsorbent particle. Once the surfactant diffuses through the swollen superabsorbent particle and reaches the outside surface, it forms a hydrophobic coating, which can be an oil-type coating, on that surface to reduce the tendency for evaporation of water therefrom. This coating reduces surface tension of the fluid on the outside surface of the superabsorbent particle to reduce the potential for water evaporation. Because the surfactant, which coats the outside surface of the superabsorbent particle after the superabsorbent particle has become substantially or fully swollen, is di-functional (that is, it has hydrophilic and hydrophobic regions), it is much more difficult for water to pass through the surfactant layer to the outside air as compared to an outer surface of a superabsorbent particle which lacks the coating. It is believed that the hydrophilic regions of the surfactant align themselves towards the interior of the superabsorbent particle where the liquid is held while the hydrophobic regions align themselves outwardly toward the air thus making it difficult for water to escape as it must pass through the hydrophobic regions prior to exiting the superabsorbent particle.

As noted above, although the surfactant material can be introduced in any area inside of the superabsorbent particle, it is desirable that the surfactant be introduced into the center of the interior of the superabsorbent particle. Such a location provides at least two benefits: (1) the superabsorbent particle is free to absorb liquid without substantial interference from the surfactant material as the surfactant material is contacted by the liquid once the superabsorbent particle is nearly fully or fully swollen; and (2) the surfactant is fully activated once the superabsorbent particle is fully or substantially fully swollen; this is desirable since evaporation from superabsorbent particles which are not fully swollen is not typically substantially problematic.

The surfactant introduced into the interior of the superabsorbent polymer can either be in liquid or in solid form, or a combination of a liquid and a solid. If the surfactant is present in liquid form in the interior of the superabsorbent particle, its diffusion from the interior of the superabsorbent particle to the outside surface of the superabsorbent particle will typically be faster as compared to a solid surfactant which must first be dissolved into the imbibed liquid before diffusion can fully occur. Regardless of whether a solid or a liquid surfactant is utilized, it is desirable that the diffusion rate of the surfactant through a superabsorbent particle swollen with a 0.9% (by weight) saline solution be such that the surfactant is sufficiently mobile upon activation to quickly migrate through the superabsorbent particle to the outer surface where it can provide the desired coating on the surface and substantially reduce evaporation of the imbibed liquid from the superabsorbent particle. It is desirable for the surfactant to be able to substantially fully diffuse to the outside surface of the superabsorbent particle in no more than about 30 minutes, and desirably no more than about 5 minutes after activation. Desirably, the diffusion rate of the surfactant is from about 30 micrometers per minute to about 150 micrometers per minute. Such a diffusion rate allows the surfactant to diffuse through the swollen superabsorbent particle at a rate sufficient to provide the intended benefit of coating the superabsorbent particle's outside surface and reducing evaporation therefrom.

Any surfactant that does not substantially interfere with the absorbing capabilities of the superabsorbent particle and that can be successfully introduced into the interior of the superabsorbent particle and provide the intended benefit of diffusing to, and coating, the outer surface of the superabsorbent particle upon swelling may be suitable for use in accordance with the present invention. Generally, it is desirable that the surfactants utilized in combination with the superabsorbent particles have an HLB in the range of from about 7 to about 12 to ensure that the surfactant is sufficiently water soluble yet will still migrate to the outer surface of a superabsorbent particle upon activation.

Typically, when the superabsorbent particle is anionic in nature (e.g. acrylate based or sulfonate based), the surfactant may be selected from non-ionic surfactants and anionic surfactants, with non-ionic surfactants being desirable. Cationic surfactants are typically less desirable when the superabsorbent particle is anionic in nature as the cationic surfactants may have excessive attraction to the superabsorbent polymer and have difficulty migrating to the surface of the swollen particle. Although anionic surfactants may be repelled by the negatively charged backbone, anionic surfactants may be suitable as substantially no reaction would occur between the polymer backbone and the surfactant.

When the superabsorbent particle is cationic in nature (e.g., a partly neutralized polyamine), the surfactant may be selected from non-ionic surfactants and cationic surfactants. With cationically charged superabsorbent particles, anionic surfactants are typically less desirable as they may tend to react with the positively charged moieties on the polymer backbone and substantially alter the superabsorbent particle rendering it less suitable for its intended purpose.

Exemplary anionic surfactants, which are suitable for use in the present invention, include fatty acids and/or fatty sulfonates or phosphates. Preferred anionic surfactants include Hostaphat CG 120, available from Clariant Functional Chemicals Corporation (Mt. Holly, N.C.). Exemplary non-ionic surfactants include fatty alcohols and ethoxylated ethanols. Suitable non-ionic surfactants include Pluronic L62 or Pluronic L43, available from BASF Corporation Performance Chemicals (Mount Olive, N.J.), and Tomadol 23-3, Tomadol 91-2.5, and Tomadol 1-5, available from Tomah Products, Inc. (Milton, Wis.). Exemplary cationic surfactants include ethoxylated amines or amides. Preferred cationic surfactants include Tomah E-14-2, available from Tomah Products, Inc.

The surfactant is desirably introduced into the interior of the superabsorbent particle in an amount sufficient such that, upon activation and migration to the outer surface of the superabsorbent particle upon the uptake of liquid by the superabsorbent particle, there is sufficient surfactant present to coat the entire outer surface to reduce evaporation from the superabsorbent particle. It is desirable that there be sufficient surfactant present to form at least a thin monolayer or skin of surfactant coating on the entire outer surface of the superabsorbent particle. Typically, a coating thickness of from about 10 Angstroms to about 60 Angstroms is desirable and will provide the intended benefit of coating the outside surface of the superabsorbent particle to reduce water evaporation from the surface of the swollen superabsorbent particle. Typically, from about 0.1% (by weight of the superabsorbent particle and surfactant) to about 1% (by weight of the superabsorbent particle and surfactant) of surfactant is a suitable amount of introduction into the superabsorbent particle. As one skilled in the art will recognize based on the disclosure herein, more or less surfactant may be required to provide the intended benefit depending upon the specific superabsorbent polymers being utilized.

Superabsorbent particles comprising a surfactant in the interior can be prepared using various manufacturing methods in accordance with the present invention. As is well known in the art, conventional superabsorbent particles can be manufactured by at least two polymerization methods, namely the solvent or solution polymerization method and the inverse suspension or emulsion polymerization method. Various methods of making conventional superabsorbent particles are set forth in U.S. Pat. Nos. 4,076,663, 4,286,082, 4,340,706, and 5,409,771, all of which, in their entirety, are incorporated by reference. Both the solvent method and the emulsion method of preparing convention superabsorbent particles can be modified in accordance with the present invention to produce superabsorbent particles comprising a surfactant in the interior.

Low evaporative superabsorbent particles comprising a surfactant in the interior can be prepared in accordance with the present invention using the modified solvent process as described herein. In the modified solvent process, low evaporative superabsorbent particles are prepared from an aqueous mixture of monomers. A poly(acrylic) acid (or other suitable starting compound) is introduced in water and dissolved to form an aqueous monomer composition. Along with the monomer, a suitable surfactant (such as, for example an anionic or non-ionic surfactant when an anionic polyacrylate is used) is introduced into the aqueous solution in the desired amount to form an aqueous solution comprising monomers and a surfactant. Finally, to the aqueous solution is added a multi-functional crosslinking agent, such as a di-functional acrylate, which substantially increases the viscosity of the solution due to the crosslinking of the monomers, and ultimately creates a thickened gel by radical polymerization. The monomeric crosslinking traps the surfactant compound in the interior of the crosslinked polymers and results in a crosslinked polymer with a surfactant in the interior. After the resulting high viscous gel material is dried, it is subjected to mechanical grinding to create a desired particle size distribution. This method of manufacturing surfactant-containing superabsorbent particles may either be carried out continuously or discontinuously to produce a superabsorbent particle composition including a surfactant in the interior of the superabsorbent particle.

Additionally, low evaporative superabsorbent particles comprising a surfactant in the interior can be prepared utilizing a modified emulsion polymerization process. In this process, an aqueous, partially neutralized acrylic acid (or other suitable compound) solution is dispersed in a hydrophobic organic solvent by means of protective colloids or emulsifiers. Also introduced into this hydrophobic organic solvent is a suitable surfactant (such as an anionic or non-ionic surfactant) in the desired amount. Polymerization is then initiated by radical initiators. After completion of the polymerization, the water is azeotropically removed from the reaction mixture and the polymeric product comprising the surfactant filtered off and dried. The cross-linking reaction may be affected by incorporating a multi-functional cross-linking agent, which is dissolved in the monomer solution, by polymerization, and/or by reacting suitable cross-linking agents with functional groups of the polymer. After crosslinking is complete, the resulting gel is dried and processed as noted above to provide the desired particles.

Further, low evaporative superabsorbent films can be prepared in accordance with the present invention. Methods of making superabsorbent films are well known in the art. In a modified process in accordance with the present invention, low evaporative superabsorbent films can be prepared by introducing a suitable surfactant in the desired amount onto the surface of one superabsorbent film, and then placing a second superabsorbent film on top of the first to form a “sandwich” of films, with the surfactant in the middle. This “sandwich” of films including a surfactant in the interior can then be utilized in an absorbent product in a manner similar to superabsorbent particles including a surfactant in the interior.

In an alternative embodiment of the present invention, low evaporative absorbent articles comprising superabsorbent particles can be prepared by incorporating a treatment agent into or onto the absorbent core, the liner, surge management layer, or other components of an absorbent article. The treatment agent, upon activation, is capable of migrating from the component to which it is applied to the outer surface of swollen superabsorbent particles also present in the absorbent core. Upon reaching the outside surface of the superabsorbent particle, the treatment agent coats that surface in a manner similar to that described above. This outer coating acts to reduce the amount of evaporation from the superabsorbent particles, and results in a reduction of the amount of humidity, and potentially malodor, within an absorbent article microenvironment as previously discussed. Although this embodiment is discussed primarily herein in combination with a fibrous network of absorbent fibers contained in the absorbent core component of a superabsorbent article, it will be recognized by one skilled in the art based on the disclosure herein that the treatment agent may be introduced onto other components of the absorbent article in a similar manner and still be capable of migrating upon activation to the outer surface of the superabsorbent particles.

As mentioned above, the absorbent core of an absorbent article typically includes a fibrous network of absorbent fibers in combination with superabsorbent particles for absorbing liquids excreted from the body such as urine, blood, menses, etc. In order to reduce humidity within the diaper microenvironment due to evaporation of water from swollen superabsorbent particles, an aqueous salt solution-soluble treatment agent can be incorporated onto the fibrous network of absorbent fibers, or other components or fibers of the absorbent core, which are typically intermixed with superabsorbent particles. Upon urination by a wearer, the treatment agent is dissolved in the urine and migrates from the fibrous network of absorbent fibers to the outside surfaces of swollen superabsorbent particles where it coats these surfaces and reduces the amount of evaporation of water therefrom to reduce overall humidity in the diaper microenvironment.

Numerous aqueous salt solution-soluble (e.g., urine soluble, menses soluble, etc.) treatment agents have been found satisfactory for incorporation onto the fibrous network of absorbent fibers for subsequent activation and migration to the superabsorbent particles. Suitable treatment agents include cationic polymers, disaccharides, and surfactants, for example. Suitable treatment agents are capable of forming specific molecular complexes with the superabsorbent polymers. The formed complexes can be electrostatic complexes or hydrogen-bonded complexes, for example. Cationic polymers can form electrostatic complexes with an anionic superabsorbent. Disaccharides or surfactants with polyether segments are believed to form hydrogen-bonded complexes with the superabsorbent polymers. The resultant coating on the superabsorbent particle is less easily permeated by water vapor than the bulk swollen superabsorbent. Specifically, effective treatment agents include cationic starch (such as, for example, Raifix 120, available from, Raisio Chemicals, a company with offices in Berwick, Pa., poly(diallyldimethyl ammonium chloride), chitosan hydrochloride, the disaccharide trehalose, cationic surfactants, anionic surfactants, and non-ionic surfactants.

These treatment agents, when incorporated onto a component of the absorbent article such as a fibrous network of absorbent fibers, for example, are sufficiently urine (or aqueous salt solution, or water) soluble to allow for dissolution into the urine from the fibers and subsequent migration to the outside surfaces of the superabsorbent particles. Because these treatment agents remain on the substrate to which they are applied (the fibrous network of absorbent fibers, for example) in a solid state, dissolution is not instantaneous, but still occurs over a relatively short period of time. While the treatment agent is dissolving into the urine excreted by the wearer and subsequently migrating toward the superabsorbent particles to coat the outer surface, the superabsorbent particles are absorbing liquid and swelling to capacity. Once the superabsorbent particles have imbibed at least some liquid, and have desirably become substantially fully swollen, the outer surface is coated by the treatment agent to reduce evaporation from the surface. Based on the disclosure herein, it will be recognized by one skilled in the art that the amount of time that the superabsorbent particles have to absorb liquid prior to coating can be controlled in part by the amount of treatment agent added to the fibrous network of absorbent fibers, or other component of the absorbent core. The more treatment agent that is added, typically the faster the coating will occur and the less time that the superabsorbent particles will have to imbibe liquid.

Without being bound to a particular theory, it is believed that the treatment agents, once dissolved into the aqueous salt solution, have a specific chemical attraction to the superabsorbent particle surface. It is believed that the treatment agent forms a substantial network of hydrogen bonds with the outer surface of the superabsorbent particle and reduces the amount of free space or void volume for absorbed liquid to pass through and evaporate. The hydrogen bonding appears to form a physical-type barrier and substantially reduces the rate of evaporation as the liquid must take a more tortuous path for evaporation as compared to an untreated surface.

The treatment agents for incorporation onto the fibrous network of absorbent fibers or other absorbent core component are introduced onto the fibers in an amount sufficient such that, upon dissolution into urine and migration to the outer surface of the superabsorbent particles, there is sufficient treatment agent present in the urine to substantially coat the entire outer surface of a substantial number of the superabsorbent particles to reduce evaporation therefrom. More specifically, the amount of treatment agent present on the fibrous network of absorbent fibers is typically from about 0.5% (by weight based on a total weight of the dry superabsorbent particles) to about 30% (by weight based on a total weight of the dry superabsorbent particles), more desirably from about 1% (by weight based on a total weight of the dry superabsorbent particles) to about 25% (by weight based on a total weight of the dry superabsorbent particles), and most desirably from about 5% (by weight based on a total weight of the dry superabsorbent particles) to about 20% (by weight based on a total weight of the dry superabsorbent particles).

The treatment agents for decreasing evaporation from superabsorbent particles may be introduced onto the fibrous network of absorbent fibers, or other fibers or components of the absorbent core, in numerous ways in accordance with the present invention to allow for their subsequent dissolution upon being contacted with a bodily fluid such as urine. For example, the treatment agent can be introduced onto the fibers during manufacturing of the fibers themselves prior to incorporating the fibers into the absorbent article, or can be introduced onto the absorbent article comprising the fibers directly. In one specific embodiment, the urine soluble treatment agents are first introduced and dissolved into water, such as deionized water, to form an aqueous treatment agent solution. The aqueous treatment agent solution is then sprayed in a fine mist or spray onto the fibrous network of absorbent fibers to achieve the desired concentration or treatment agent on the fibers. After misting or spraying is discontinued, the treated fibers are subjected to a drying process to drive off the water and leave the treatment agent on the fibers. After drying, the fibers may be incorporated into the absorbent core of the article in the desired amount. Once the fibers are dried, the treatment agent remains on the fibers until activation by contact with urine or another salt-containing bodily fluid or water.

Alternatively, the treatment agents may be introduced into the fibrous network of absorbent fibers directly and remain loose in the absorbent core until urination occurs at which time they will be dissolved and migrate to the outer surface of the superabsorbent particles. Because the fibrous network of absorbent fibers is typically formed tightly and intermixed with superabsorbent particles, the treatment agents will generally remain in the fibrous network of absorbent fibers in the absorbent core until urination. Alternatively, if the treatment agent is a liquid, it may be sprayed directly onto the absorbent core in the desired concentration and allowed to dry.

The present invention is illustrated in the following Example, which is in no way intended to limit the scope of the present invention.

### EXAMPLE 1

In this Example several treatment agents at concentrations varying from 1.25% to 30% (by weight based on the dry weight of the superabsorbent particles) were introduced onto superabsorbent particles swollen with a 0.9% (by weight) saline solution and evaluated for their ability to reduce the amount of water evaporation from the swollen superabsorbent particles.

The following treatment agents at the following concentrations were evaluated in this Example: (1) Trehalose dihydrate (5%); (2) Poly(diallyldimethyl ammonium chloride) (10%); (3) Cationic starch (20%); and (4) Chitosan hydrochloride (1.25%). Each treatment agent was dissolved, at the above-noted concentration, into a different aqueous solution comprising 0.9% (by weight) sodium chloride (synthetic urine).

Once the treatment agent was completely dissolved into the 0.9% (by weight) saline solution, the solution was used to saturate superabsorbent particles (FAVOR 880, Stockhausen, Inc., Greensboro, N.C.) to a level of 25 grams of solution per gram of superabsorbent particles. After saturation, the swollen superabsorbent particles were introduced into an enclosed container fitted with a humidity gauge and conditioned within an oven at a temperature of about 35° C. for a period of eight hours. Humidity readings were taken as a function of time for eight hours at the following times: (1) 0.1 hours; (2) 0.5 hours; (3) 1 hour; (4) 1.5 hours; (5) 2 hours; (6) 2.5 hours; (7) 3 hours; (8) 3.5 hours; (9) 4 hours; and (10) 8 hours. FAVOR 880 saturated with 0.9% sodium chloride solution to a level of 25 grams of solution per gram of superabsorbent was used as a control. The percent relative humidity within the test chamber for the control and each of the treatment agents at various times is shown in Table 1.

TABLE 1

| Treatment | | | | | | | | | | |
|---|---|---|---|---|---|---|---|---|---|---|
| Agent | 0.1 Hr. | 0.5 Hr. | 1 Hr. | 1.5 Hr. | 2 Hrs. | 2.5 Hrs. | 3 Hrs. | 3.5 Hrs. | 4 Hrs. | 8 Hrs. |
| None | 28.4% | 46.8% | 54.3% | 57.8% | 60.1% | 61.6% | 62.7% | 63.5% | 64.1% | 66.1% |
| Trehalose | 11.0% | 25.1% | 34.9% | 40.9% | 45.8% | 49.2% | 51.7% | 53.7% | 55.2% | 61.7% |
| dihydrate | | | | | | | | | | |
| Poly(diallyldi- | 16.5% | 30.8% | 39.3% | 45.3% | 49.5% | 52.5% | 54.8% | 56.5% | 57.9% | 63.5% |
| methyl | | | | | | | | | | |
| ammonium | | | | | | | | | | |
| chloride) | | | | | | | | | | |
| Cationic Starch | 11.7% | 37.4% | 45.8% | 49.9% | 52.4% | 54.2% | 55.6% | 56.8% | 57.7% | 61.6% |
| Chitosan | 13.8% | 30.3% | 39.3% | 45.3% | 49.5% | 52.6% | 54.7% | 56.3% | 57.6% | 62.7% |
| Hydrochloride | | | | | | | | | | |

As the data in Table 1 indicate, each of the treatment agents substantially reduced the amount of water evaporation from the superabsorbent particles at each time point. Notably, each treatment agent at the 0.1 hours and 0.5 hour time point substantially reduced the amount of evaporation of water from the superabsorbent particles as compared to the untreated control. These early time points are particularly important as fully loaded absorbent articles, such as diapers, typically will not remain on the body for more than about 30 minutes or 1 hour and as such, a substantial decrease in evaporation during this time period is desirable.

In view of the above, it will be seen that the several objects of the invention are achieved. As various changes could be made in the above-described products and methods without departing from the scope of the invention, it is intended that all matter contained in the above description be interpreted as illustrative and not in a limiting sense.

What is claimed is:

1. A low evaporative absorbent article comprising a superabsorbent particle and a treatment agent, the treatment agent being capable of migrating to and coating an outer surface of the superabsorbent particle with an evaporation-reducing coating upon being contacted with an aqueous salt solution.

2. The low evaporative absorbent article as set forth in claim 1 wherein the aqueous salt solution is selected from the group consisting of urine and menses.

3. The low evaporative absorbent article as set forth in claim 1 wherein the treatment agent is located within the superabsorbent particle.

4. The low evaporative absorbent article as set forth in claim 3 wherein the treatment agent is located at the center of the superabsorbent particle.

5. The low evaporative absorbent article as set forth in claim 3 wherein the treatment agent is a surfactant.

6. The low evaporative absorbent article as set forth in claim 5 wherein the surfactant has an HLB in the range of from about 7 to about 12.

7. The low evaporative absorbent article as set forth in claim 5 wherein the treatment agent is a solid surfactant.

8. The low evaporative absorbent article as set forth in claim 5 wherein the treatment agent is a liquid surfactant.

9. The low evaporative absorbent article as set forth in claim 5 wherein the treatment agent comprises an anionic surfactant.

10. The low evaporative absorbent article as set forth in claim 9 wherein the treatment agent comprises an anionic surfactant selected from the group consisting of fatty acids, fatty sulfonates, and fatty phosphates.

11. The low evaporative absorbent article as set forth in claim 9 wherein the anionic surfactant is Hostaphat CG 120.

12. The low evaporative absorbent article as set forth in claim 5 wherein the treatment agent comprises a non-ionic surfactant.

13. The low evaporative absorbent article as set forth in claim 12 wherein the non-ionic surfactant is selected from the group consisting of fatty alcohols and ethoxylated ethanols.

14. The low evaporative absorbent article as set forth in claim 12 wherein the non-ionic surfactant is selected from the group consisting of Pluronic L62, Pluronic L43, Tomadol 23-3, Tomadol 91-2.5, and Tomadol 1-5.

15. The low evaporative absorbent article as set forth in claim 5 wherein the treatment agent comprises a cationic surfactant.

16. The low evaporative absorbent article as set forth in claim 15 wherein the cationic surfactant is selected from the group consisting of ethoxylated amines or amides.

17. The low evaporative absorbent article as set forth in claim 15 wherein the cationic surfactant is Tomah E-14-2.

18. The low evaporative absorbent article as set forth in claim 3 wherein the treatment agent is present within the superabsorbent particle in an amount of from about 0.1% (by weight of the superabsorbent particle and surfactant) to about 1.0% (by weight of the superabsorbent particle and surfactant).

19. The low evaporative absorbent article as set forth in claim 3 wherein the treatment agent has a rate of diffusion through the superabsorbent particle of from about 30 micrometers per minute to about 150 micrometers per minute.

20. The low evaporative absorbent article as set forth in claim 1 wherein the treatment agent is located outside of the superabsorbent particle.

21. The low evaporative absorbent article as set forth in claim 20 further comprising a fibrous network of absorbent fibers.

22. The low evaporative absorbent article as set forth in claim 21 wherein the treatment agent is located on the surface of the fibrous network of absorbent fibers.

23. The low evaporative absorbent article as set forth in claim 22 wherein the treatment agent is selected from the group consisting of water-soluble cationic polymers and water-soluble disaccharides.

24. The low evaporative absorbent article as set forth in claim 22 wherein the treatment agent is selected from the group consisting of cationic starch, poly(diallyldimethyl ammonium chloride), chitosan hydrochloride, and trehalose.

25. The low evaporative absorbent article as set forth in claim 22 wherein the treatment agent is present in the low evaporative absorbent article in an amount of from about 0.5% (by weight based on the total weight of the superabsorbent particles in the article) to about 30% (by weight based on the total weight of the superabsorbent particles in the article).

26. The low evaporative absorbent article as set forth in claim 22 wherein the treatment agent is present in the low evaporative absorbent article in an amount of from about 1% (by weight based on the total weight of the superabsorbent particles in the article) to about 25% (by weight based on the total weight of the superabsorbent particles in the article).

27. The low evaporative absorbent article as set forth in claim 22 wherein the treatment agent is present in the low evaporative absorbent article in an amount of from about 5% (by weight based on the total weight of the superabsorbent particles in the article) to about 20% (by weight based on the total weight of the superabsorbent particles in the particle).

28. A method for controlling skin overhydration caused by water evaporation from a superabsorbent particle contained in an absorbent product, the method comprising:
introducing a surfactant into the interior of the superabsorbent particle such that when the superabsorbent particle is contacted with a salt containing solution and swells, the surfactant diffuses to the outer surface of the superabsorbent particle and coats the outer surface of the superabsorbent particle with an evaporation reducing coating; and
contacting the superabsorbent particle with an aqueous salt solution thereby allowing the superabsorbent particle to swell and the surfactant to diffuse to and coat the outer surface of the superabsorbent particle.

29. The method as set forth in claim 28 wherein the aqueous salt solution is selected from the group consisting of urine and menses.

30. The method as set forth in claim 28 wherein the surfactant is introduced into the center of the superabsorbent particle.

31. The method as set forth in claim 28 wherein the surfactant is an anionic surfactant.

32. The method as set forth in claim 31 wherein the anionic surfactant is selected from the group consisting of fatty acids, fatty sulfonates, and fatty phosphates.

33. The method as set forth in claim 31 wherein the anionic surfactant is Hostaphat CG 120.

34. The method as set forth in claim 28 wherein the surfactant is a non-ionic surfactant.

35. The method as set forth in claim 34 wherein the non-ionic surfactant is selected from the group consisting of fatty alcohols and ethoxylated ethanols.

36. The method as set forth in claim 34 wherein the non-ionic surfactant is selected from the group consisting of Pluronic L62, Pluronic L43, Tomadol 23-3, Tomadol 91-2.5, and Tomadol 1-5.

37. The method as set forth in claim 28 wherein the surfactant is a cationic surfactant.

38. The method as set forth in claim 37 wherein the cationic surfactant is selected from the group consisting of ethoxylated amines or amides.

39. The method as set forth in claim 37 wherein the cationic surfactant is Tomah E-14-2.

40. The method as set forth in claim 30 wherein the surfactant is present within the superabsorbent particle in an amount of from about 0.1% (by weight based on the total weight of the superabsorbent particle) to about 1.0% (by weight based on the total weight of the superabsorbent particle).

41. The method as set forth in claim 30 wherein the surfactant has a rate of diffusion through the superabsorbent particle of from about 30 micrometers per minute to about 150 micrometers per minute.

42. A method for controlling skin overhydration caused by water evaporation from a superabsorbent particle contained in an absorbent product, the method comprising:
introducing a treatment agent onto a fibrous network of absorbent fibers comprising the superabsorbent particle such that when the fibrous network of absorbent fibers is contacted with an aqueous salt solution, the treatment agent diffuses from the fibrous network of absorbent fibers to the outer surface of the superabsorbent particle and coats the outer surface of the superabsorbent particle with an evaporation-reducing coating; and
contacting the fibrous network of absorbent fibers with an aqueous salt solution thereby allowing the treatment agent to diffuse to and coat the outer surface of the superabsorbent particle.

43. The method as set forth in claim 42 wherein the aqueous salt solution is selected from the group consisting of urine and menses.

44. The method as set forth in claim 42 wherein the treatment agent is selected from the group consisting of water-soluble cationic polymers and water-soluble disaccharides.

45. The method as set forth in claim 42 wherein the treatment agent is selected from the group consisting of cationic starch, poly(diallyldimethyl ammonium chloride), chitosan hydrochloride, and trehalose.

46. The method as set forth in claim 42 wherein the treatment agent is present on the fibrous network of absorbent fibers in an amount of from about 0.5% (by weight based on the total weight of the superabsorbent particles) to about 30% (by weight based on the total weight of the superabsorbent particles).

47. The method as set forth in claim 42 wherein the treatment agent is present on the fibrous network of absorbent fibers in an amount of from about 1% (by weight based on the total weight of the superabsorbent particles) to about 25% (by weight based on the total weight of the superabsorbent particles).

48. The method as set forth in claim 42 wherein the treatment agent is present on the fibrous network of absorbent fibers in an amount of from about 5% (by weight based on the total weight of the superabsorbent particles) to about 20% (by weight based on the total weight of the superabsorbent particles).

49. A superabsorbent composite particle comprising a superabsorbent polymer and a treatment agent, the treatment agent being located inside of the superabsorbent polymer and being capable of diffusing to and coating the outside surface of the superabsorbent polymer with an evaporation-reducing coating upon being contacted with an aqueous salt solution.

50. The superabsorbent composite particle as set forth in claim 49 wherein the treatment agent is a surfactant.

51. The superabsorbent composite particle as set forth in claim 50 wherein the treatment agent is a solid surfactant.

52. The superabsorbent composite particle as set forth in claim 50 wherein the treatment agent is a liquid surfactant.

53. The superabsorbent composite particle as set forth in claim 50 wherein the surfactant comprises an anionic surfactant.

54. The superabsorbent composite particle as set forth in claim 53 wherein the treatment agent comprises an anionic surfactant selected from the group consisting of fatty acids, fatty sulfonates, and fatty phosphates.

55. The superabsorbent composite particle as set forth in claim 53 wherein the anionic surfactant is Hostaphat CG 120.

56. The superabsorbent composite particle as set forth in claim 50 wherein the treatment agent comprises a non-ionic surfactant.

57. The superabsorbent composite particle as set forth in claim 56 wherein the non-ionic surfactant is selected from the group consisting of fatty alcohols and ethoxylated ethanols.

58. The superabsorbent composite particle as set forth in claim 56 wherein the non-ionic surfactant is selected from the group consisting of Pluronic L62, Pluronic L43, Tomadol 23-3, Tomadol 91-2.5, and Tomadol 1-5.

59. The superabsorbent composite particle as set forth in claim 50 wherein the treatment agent comprises a cationic surfactant.

60. The superabsorbent composite particle as set forth in claim 59 wherein the cationic surfactant is selected from the group consisting of ethoxylated amines or amides.

61. The superabsorbent composite particle as set forth in claim 59 wherein the cationic surfactant is Tomah E-14-2.

62. The superabsorbent composite particle as set forth in claim 50 wherein the surfactant is present within the superabsorbent polymer in an amount of from about 0.1% (by weight of the superabsorbent polymer and surfactant) to about 1.0% (by weight of the superabsorbent polymer and surfactant).

63. The superabsorbent composite particle as set forth in claim 50 wherein the surfactant has a rate of diffusion through the superabsorbent polymer of from about 30 micrometers per minute to about 150 micrometers per minute.

64. A method of preparing a low evaporative superabsorbent composite comprising a superabsorbent particle and a surfactant, the method comprising:
introducing an acrylate based or sulfonate based monomer and a surfactant into water to form an aqueous monomer and surfactant solution;
introducing a multi-functional crosslinking agent into the aqueous monomer and surfactant solution, the multi-functional crosslinking agent increasing the viscosity of the aqueous solution and causing a gel to form by radical polymerization;
drying the gel; and
subjecting the dried gel to a grinding process to form superabsorbent particles.

65. A method for reducing evaporative potential of an absorbent article comprising introducing a surfactant into a superabsorbent particle carried by the absorbent article such that upon contact of the superabsorbent particle with a salt-containing solution, the surfactant diffuses to an outer surface of the superabsorbent particle to coat the outer surface with an evaporation-reducing coating.
